(19) United States
(12) Patent Application Publication
(10) Pub. No.: US 2025/0276144 A1
(43) Pub. Date: Sep. 4, 2025

(54) SYSTEMS AND METHODS FOR DEPLOYING AND SECURING ENDOTRACHEAL TUBES

(71) Applicant: THE PENN STATE RESEARCH FOUNDATION, University Park, PA (US)

(72) Inventors: Sprague W. Hazard, III, University Park, PA (US); David Goldenberg, University Park, PA (US); Johnathan McGinn, University Park, PA (US); Neerav Goyal, University Park, PA (US)

(21) Appl. No.: 18/854,491

(22) PCT Filed: Apr. 6, 2023
(86) PCT No.: PCT/US2023/017728

§ 371 Date: Oct. 4, 2024

Related U.S. Application Data
(60) Provisional application No. 63/328,946, filed on Apr. 8, 2022.

(51) Int. Cl. A61M 16/04

(52) U.S. Cl. CPC A61M 16/0497; A61M 16/0402; A61M 16/0434

(57) ABSTRACT

An endotracheal tube can include a tube having a distal end that is configured to be inserted into the trachea of a patient and a proximal end that is configured to be positioned within the mouth of the patient or outside of the patient. The endotracheal tube can include a tether having a first end coupled to the tube, and a magnet coupled to the tether. The magnet can be configured to be advanced through the pharynx, through and out the nasal cavity thereby positioning the soft palate of the patient between the tube and the tether.

### CROSS-REFERENCE TO RELATED APPLICATIONS

This application claims the benefit of and priority to U.S. Provisional Application Ser. No. 63/328,946, filed on Apr. 8, 2022, the entirety of which is incorporated by reference herein.

### STATEMENT REGARDING FEDERALLY SPONSORED RESEARCH

N/A.

### BACKGROUND

Endotracheal tubes are typically deployed in medical situations in which the patient is unable to breathe (or is having difficulty breathing) on his/her own, which can occur during general anesthesia procedures in which anesthesia temporarily paralyzes the breathing muscles including the diaphragm, or during other scenarios in which the patient is having difficulty breathing (e.g., due to respiratory diseases, pneumothorax, heart failure, overdose, stroke, etc.), etc.

While the use of endotracheal tubes can be extremely helpful in establishing and maintaining a patient's airway, in some cases, patients can inadvertently remove the endotracheal tube, thereby compromising the established airway. For example, after waking from a sedated state, a confused and startled patient may improperly believe that the endotracheal tube is choking them, when in fact, the endotracheal tube is allowing the patient to breathe. Thus, such a patient may tug at the endotracheal tube with the aim of removing the tube, which can not only dislodge the endotracheal tube (e.g., requiring redeployment of the endotracheal tube, which undesirably cause patient discomfort and consume additional hospital resources including materials and practitioner expertise), but can even cause death if, for example, the airway is unable to be reestablished within a certain amount of time.

Thus, it would be desirable to have improved systems and methods for deploying and securing endotracheal tubes.

### SUMMARY OF THE DISCLOSURE

Some embodiments of the disclosure provide an endotracheal tube. The endotracheal tube may include a tube having a distal end that is configured to be inserted into the trachea of a patient and a proximal end that is configured to be positioned within the mouth of the patient or outside of the patient; a tether having a first end coupled to the tube; and a magnet coupled to the tether, the magnet being configured to be advanced through the pharynx, through and out the nasal cavity thereby positioning the soft palate of the patient between the tube and the tether.

In certain embodiments of the endotracheal tube, when the proximal end of the tube is advanced away from the patient, the tether may contact the soft palate to block further advancement of the proximal end of the tube away from the patient.

In various embodiments, the endotracheal tube may further include a channel that extends along the tube; a slide coupled to a second end of the tether opposite the first end; and a stop coupled to the tube, where the magnet may be configured to be moved along the channel in a first direction towards the proximal end of the tube until the slide contacts the stop thereby blocking further advancement of the magnet in the first direction.

In particular embodiments of the endotracheal tube, a second end of the tether opposite the first end may be configured to be coupled to an anchor that is external to the patient thereby fixing the position of the endotracheal tube within the patient.

In some embodiments of the endotracheal tube, the anchor may wrap around the head of the patient, and a portion of the tube may be coupled to the anchor.

In certain embodiments of the endotracheal tube, the magnet may be removably coupled to the tether.

Particular embodiments of the endotracheal tube may further include a first adapter coupled to the proximal end of the tube, a second adapter of a conduit in fluid communication with a ventilator may be configured to be removably coupled to the first adapter, and the first adapter may be configured to be positioned within the mouth of the patient when the distal end of the tube is placed in the trachea of the patient.

In various embodiments of the endotracheal tube, when the second adapter is pulled away from the first adapter of the tube, the second adapter may decouple from the first adapter.

In some embodiments of the endotracheal tube, the magnet may be a first magnet, the first adapter may include a second magnet, the second adapter may include a third magnet, and the first adapter may magnetically couple to the second adapter, via the interaction between the second magnet and the third magnet.

Certain embodiments of the endotracheal tube may further include an inflatable cuff coupled to the tube which may be configured to be positioned within the trachea of the patient, and when the first adapter is coupled to second adapter and the second adapter is pulled away from the first adapter, the second adapter may decouple from the first adapter with the distal end of the tube remaining in the trachea.

In various embodiments of the endotracheal tube, the magnet may be a first magnet, and a probe with a second magnet coupled thereto may be configured to be inserted into the nasal cavity, through the nasal cavity, into the pharynx to be magnetically coupled to the first magnet and, with the second magnet magnetically coupled to the first magnet, the probe may be configured to be retreated back through and out the nasal cavity.

Some embodiments of the disclosure provide a method for deploying an endotracheal tube. The methods may include inserting a distal end of a tube into the trachea of a patient; positioning a proximal end of the tube within the mouth of the patient or outside of the patient, a first end of a tether being coupled to the tube, and a magnet being coupled to the tether; and advancing the magnet through the pharynx of the patient, through and out the nasal cavity such that the soft palate of the patient is positioned between the tube and the tether.

In certain embodiments of the method, when the proximal end of the tube is advanced away from the patient, the method may further include contacting the soft palate with the tether to block further advancement of the proximal end of the tube away from the patient.

Various embodiments of the method may further include: providing a channel that extends along the tube, where a slide may be coupled to a second end of the tether opposite the first end and a stop may be coupled to the tube; and moving the magnet along the channel in a first direction towards the proximal end of the tube until the slide contacts the stop to block further advancement of the magnet in the first direction.

In particular embodiments, the method may further include coupling a second end of the tether opposite the first end to an anchor that is external to the patient to fix the position of the endotracheal tube within the patient.

In some embodiments, the method may further include wrapping the anchor around the head of the patient, and coupling a portion of the tube to the anchor.

In various embodiments of the method, the magnet may be removably coupled to the tether.

Certain embodiments of the method may further include: providing a first adapter coupled to the proximal end of the tube, where a second adapter of a conduit in fluid communication with a ventilator may be configured to be removably coupled to the first adapter; and positioning the first adapter within the mouth of the patient when the distal end of the tube is placed in the trachea of the patient.

In particular embodiments of the method, when the second adapter is pulled away from the first adapter of the tube, the method may further include decoupling the second adapter from the first adapter.

In some embodiments of the method, the magnet may be a first magnet, wherein the first adapter may include a second magnet, the second adapter may include a third magnet, where the method may further include magnetically coupling the first adapter to the second adapter based on the interaction between the second magnet and the third magnet.

Various embodiments of the method may further include: providing an inflatable cuff coupled to the tube, positioning the inflatable cuff within the trachea of the patient, coupling the first adapter to the second adapter, and decoupling, when the second adapter is pulled away from the first adapter, the second adapter from the first adapter, wherein the distal end of the tube may remain in the trachea.

In particular embodiments of the method, the magnet may be a first magnet and the method may further include: inserting a probe with a second magnet coupled thereto into the nasal cavity, through the nasal cavity, and into the pharynx, magnetically coupling the second magnet to the first magnet, and retreating the probe with the second magnet magnetically coupled to the first magnet back through and out the nasal cavity.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of an endotracheal tube.

FIG. 2 shows a probe that is configured to retrieve the magnet of the endotracheal tube of FIG. 1 depicted in a straight configuration.

FIG. 3 shows the probe of FIG. 2 depicted in a curved configuration.

FIG. 4 shows a schematic illustration of another endotracheal tube.

FIG. 5 shows a schematic illustration of the endotracheal tube of FIG. 1 deployed within the patient.

FIG. 6 shows a schematic illustration of an endotracheal tube system with adapters of the endotracheal tube system separated from each other.

FIG. 7 shows a schematic illustration of the endotracheal tube system of FIG. 7 with the adapters coupled together.

FIG. 8 shows a schematic illustration of another endotracheal tube system.

FIG. 9 shows a schematic illustration of another endotracheal tube system.

### DETAILED DESCRIPTION OF THE PRESENT DISCLOSURE

As described above, a patient may inadvertently remove a previously deployed endotracheal tube (i.e., an unplanned extubation). Unplanned extubations occur frequently in the medical setting, and have high mortality risks associated therewith. However, there are few (if any) devices available that can mitigate, or deter removal of, a deployed endotracheal tube to prevent an unplanned extubation and many that are in existence rely on affixing an appliance to the skin and connecting/adhering this appliance to the outer lumen of the endotracheal tube

Some embodiments of the disclosure provide advantages for addressing these and other issues by providing improved systems and methods for deploying and securing endotracheal tubes. For example, some embodiments of the disclosure provide an endotracheal tube that can behaviorally deter removal of the endotracheal tube (e.g., by the patient with the endotracheal tube deployed therein), and can further secure the endotracheal tube relative to the patient, both of which can decrease the likelihood of an unplanned extubation (e.g., in an intensive care unit setting or in the operating room where mechanical forces are inadvertently applied to the tube directly through movement of the patient thereby leading to removal of the endotracheal tube. As a more specific example, the endotracheal tube can include a tube, a tether with one end coupled to the tube, and a magnet coupled to the tether. After the endotracheal tube is positioned in the trachea of the patient, a (flexible) probe with a magnet coupled thereto can be inserted into the nasal cavity, through the nasal cavity, into the pharynx, and the magnet of the probe can be magnetically coupled to the magnet of the endotracheal tube. Then, the probe with the magnets coupled together, can be retracted back through the nasal cavity and out of the patient, and the tether can be secured to the patient (e.g., an anchor, such as, for example, a tube holder). In this way, because the tether is coupled to the endotracheal tube, securing the tether outside of the patient further secures the endotracheal tube (e.g., by blocking pulling of the endotracheal tube out of the patient), while deterring the patient from removing the endotracheal tube. For example, when the endotracheal tube is deployed, the tube is positioned within the trachea, while the tether extends along the pharynx, through the nasal cavity, and out of the nasal cavity. Thus, the soft and hard palate are positioned between the tether and the tube. In this way, if the endotracheal tube is pulled away from the patient, the tether contacts the soft palate, which deters the patient from pulling the endotracheal tube further away (e.g., because this can be uncomfortable, or can cause pain and the hard palate is rigid and will provide a fixation point for the tethering apparatus described above).

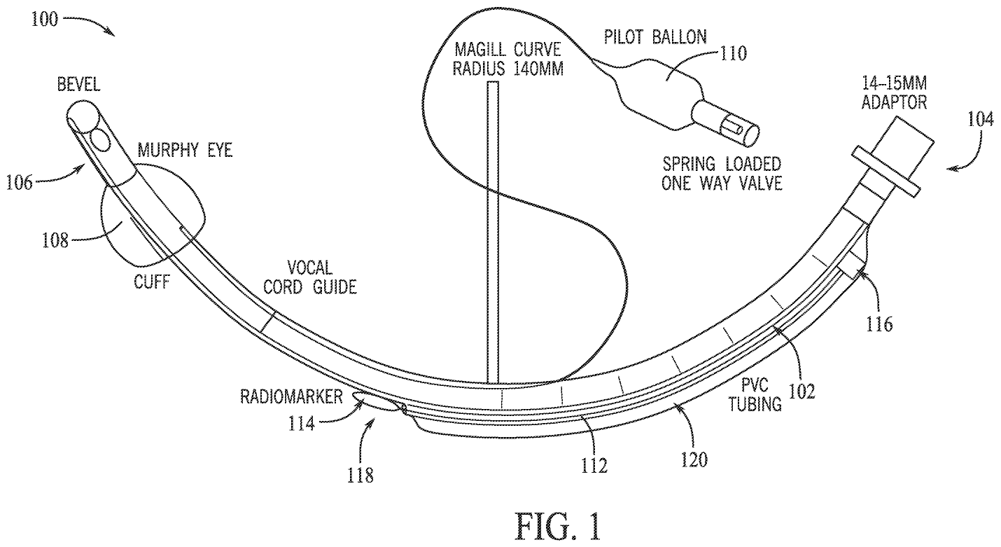

FIG. 1 shows a schematic illustration of an endotracheal tube 100. The endotracheal tube 100 can include a tube 102 having a proximal end 104 and a distal end 106 opposite the proximal end 104, an inflatable cuff 108 coupled to the tube 102, a pilot balloon 110 in fluid communication with the inflatable cuff 108, a tether 112, a magnet 114, a slide 116, a stop 118, and a channel 120. In some embodiments, the distal end 106 of the tube 102 can be inserted into the mouth of the patient, and advanced until the distal end 106 reaches the trachea of the patient. At this point, the proximal end 104 of the tube 102 can be positioned outside of the patient, or within the mouth of the patient. As shown in FIG. 1, the inflatable cuff 108 can be coupled to the tube 102 at (or proximal to) the distal end 106 of the tube 102. In this way, when the inflatable cuff 108 is inflated, the inflatable cuff 108 contacts the trachea to secure the tube 102 (and in particular the distal end 106 of the tube 102) within the trachea. In some cases, the inflatable cuff 108 can extend entirely around the tube 102, and the inflatable cuff 108 can be selectively inflated via a fluid source (e.g., a pump, such as an air pump). For example, fluid from a fluid source can be driven through the pilot balloon 110 and into the inflatable cuff 108 to inflate the inflatable cuff 108. In some cases, the pilot balloon 110 can provide a visual indication as to the degree to which the inflatable cuff 108 is inflated. For example, the greater the inflation of the pilot balloon 110, the greater the inflation of the inflatable cuff 108.

As shown in FIG. 1, the magnet 114 is coupled to the tether 112, and the slide 116 is coupled to the tether 112. In particular, the magnet 114 is coupled to one end of the tether 112 and the slide 116 is coupled to the other end of the tether 112. In other configurations, however, the magnet 114 and the slide 116 can be coupled to other portions of the tether 112. For example, the magnet 114 can be coupled to the tether 112 away from either end of the tether 112 (e.g., centrally located on the tether 112), while the slide 116 can be coupled to the tether 112 away from the other end of the tether 112. Regardless of the configuration, the magnet 114 and the slide 116 can be coupled to the tether 112 so that the magnet 114 is separated from the slide 116. As shown in FIG. 1, the tether 112 with the magnet 114 and the slide 116 coupled thereto, can be positioned within a channel 120. In some cases, the wall that defines the channel 120 can be coupled to the outside of the tube 102, or inside of the tube 102 (e.g., within the lumen of the tube 102). In other cases, the channel 120 can be directed through a wall of the tube 102. In other cases, the endotracheal tube 100 does not include the channel 120, and in other cases, the magnet 114 can be positioned outside of the channel 120 with the slide 116 positioned within the channel 120.

The stop 118 serves to hold the slide 116 onto the tube 102 (e.g., at the end of the channel 120) in order to anchor the tether 112 to the tube 102. In some embodiments, the stop 118 can be coupled to the tube 102 and can be positioned at one end of the channel 120, which can be closer to the distal end 106 of the tube 102. For example, depending on the anchor location on the tube 102, the position of the stop 118 can be moved to, for example, accommodate differences in anatomical features (e.g., the size of the mouth). In this regard, for example, the stop 118 can be slideably engaged within the channel 120 to adjust the position of the stop 118 relative to the tube 102 to accommodate for differing anatomies (e.g., to ensure that the anchor point is within the trachea).

The stop 118 can be implemented in different ways. For example, the stop 118 can be a hole, a protrusion, etc. Regardless of the configuration, when the magnet 114 is pulled (e.g., by another magnet), the magnet 114 can be pulled in an opposing direction, until the slide 116 reaches and contacts the stop 118. At this point, further puling of the magnet 114 will be blocked due to the stop 118 contacting and thus blocking movement of the slide 116 in the direction of the pulling. For example, as the magnet 114 is pulled, the tether 112 passes through (or by) the stop 118 (e.g., due to the size of tether being smaller than the stop 118), however, the slide 116 is larger than the stop 118, and thus the stop 118 is unable to move past the stop 118 in the direction opposite to the pulling direction.

The magnet 114 can be implemented in different ways. For example, the magnet 114 can be a single magnet or a group of magnets. For example, the magnet 114 can be an array of individual magnets that are coupled together (e.g., a row of magnets, a 2-D array of magnets, etc.). In this way, a probe that includes a magnet can more easily locate the array of magnets that make up the magnet 114 at least because the array of magnets takes up a larger extent of the tube 102, and can provide a greater attractive force to magnetically attract a magnet that is associated with the probe. In some cases, the magnet 114 can be a permanent magnet, such as, for example, a rare-earth magnet (e.g., a neodymium magnet). The slide 116 can also be implemented in different ways. For example, the slide 116 can have different shapes (e.g., a prism, a sphere, an ellipse, etc.). In some cases, the slide 116 having one or more curves (e.g., the slide 116 being a sphere) can be advantageous in that the slide 116 can more easily slide within the channel 120, and the slide 116 is less likely to undesirably puncture the wall of the channel 120 when the slide 116 has one or more curves. In some cases, the shape of the slide 116 can be complementary to the shape of the stop 118. For example, the slide 116 can be convex (or concave), while the stop 118 can be concave (or convex). In this way, the slide 116 can more easily interface with the stop 118.

In some embodiments, the magnet 114 can be removably coupled to the tether 112, or can be removable from the tether 112. For example, the tether 112 can include a region of material weakness (e.g., a perforation, a tear, a region having a smaller thickness, etc.), that can facilitate easier removal of the magnet from the tether 112. In this way, the magnet 114 can, after being removed from the patient while still coupled to the tether 112, be subsequently removed from the tether 112 to ensure that the magnet 114 has been removed from the patient. In addition, the magnet 114 having been removed, can be cleaned (e.g., autoclaved), recycled, etc., which can be advantageous when the magnet 114 is a rare-earth magnet (e.g., a magnet that is costly).

In some embodiments, the tether 112 (e.g., in an uncoiled state) can be longer than the channel 120. In some configurations, the tether 112 can transition from a compacted state (e.g., a coiled state) to an unfurled state. In this way, as the magnet 114 is moved, the tether 112 transitions from the compacted state to the unfurled state, without moving the slide 116 substantially (or at all). In some cases, although not shown in FIG. 1, the slide 116 can be in contact with the stop 118 before moving the magnet 114 and unraveling of the tether 112. In this way, as the magnet 114 is moved (e.g., pulled), the magnet 114 unravels the tether 112 until the tether 112 becomes completely unraveled. At this point, further pulling of the magnet 114 can cause the tether 112 to be tensilely loaded because the slide 116 is blocked by the stop 118. This loading of the tether 112 can be felt by the practitioner, which can provide a tactile response for the practitioner to stop pulling the magnet 114.

In some embodiments, the slide 116 and the magnet 114 can be replaced. In this case, the magnet 114 can be positioned where the slide 116 is in FIG. 1, and the slide 116 can be positioned where the magnet 114 is in FIG. 1. Thus, the magnet 114 can be positioned closer to the proximal end 104 of the endotracheal tube 102 than the slide 116. Correspondingly, the mechanical stop 118 can be positioned at the opposing end of the channel 120 (e.g., so that the mechanical stop 118 is closer to the proximal end 104 than the distal end 106 of the endotracheal tube 102). In this scenario, including when the magnet 114 is pulled (e.g., by another magnet) in a first direction, the slide 116 is also pulled in the first direction, until the slide 116 reaches and contacts the stop 118. At this point, further puling of the magnet 114 will be blocked due to the stop 118 contacting and thus blocking movement of the slide 116 in the direction of the pulling. For example, as the magnet 114 is pulled, the tether 112 passes through (or by) the stop 118 (e.g., due to the size of tether being smaller than the stop 118), however, the slide 116 is larger than the stop 118, and thus the stop 118 is unable to move past the stop 118 in the pulling direction.

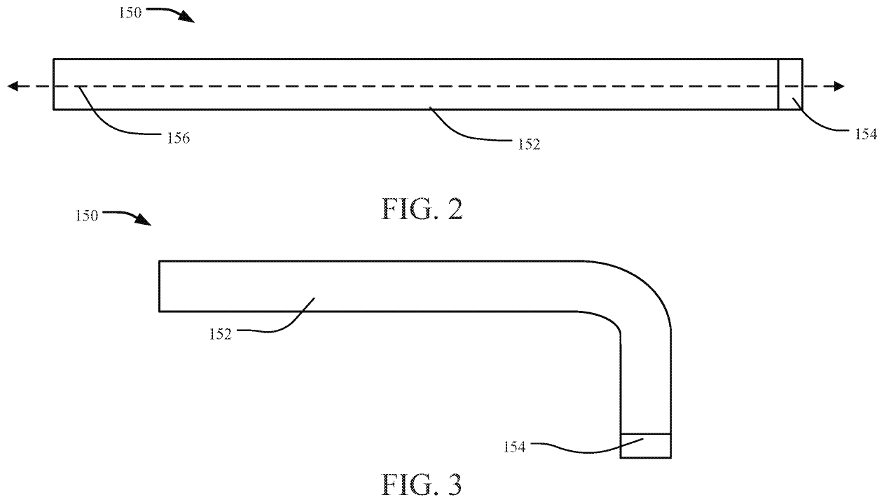

FIG. 2 shows a probe 150 that is configured to retrieve the magnet 114 with the probe 150 being depicted in a straight configuration, while FIG. 3 shows the probe 150 in a curved configuration. The probe 150 can include a body 152, and a magnet 154 coupled to the body 152. As shown in FIG. 2, the magnet 154 can be coupled to a distal end of the body 152, integrated within the body 152 of the probe 150, etc. The magnet 154 can be implemented in a similar manner as the magnet 114 of the endotracheal tube 100. For example, the magnet 154 can be a permanent magnet (e.g., a rare-earth magnet), and can be a single magnet, or multiple individual magnets coupled together. For example, in some cases, the magnet 154 can have multiple pairs of opposite poles (e.g., a north pole and a south pole), with each pair of poles being oriented in the same direction. In some cases, the magnet 154 can define an axis that extends through both poles of the magnet 154, and the axis can be substantially (i.e., deviating by less than 10 percent from) parallel, or perpendicular to a length of the tube 102. In some cases, the axis being substantially parallel to a longitudinal axis 156 of the body 152 can be advantageous in that the pole of the magnet 154 can be more in alignment with movement of the probe 150, which can facilitate better coupling between the magnet 154 and the magnet 114.

FIG. 3 shows the distal end of the body 152 including the magnet 154 being oriented away from the proximal end of the body 152 (e.g., the distal end and the proximal end of the body 152 are out of alignment with each other). The probe 150 is configured to curve to be oriented in different ways, so as to traverse around different structures within the nasal cavity, the pharynx, and the trachea. For example, the body 152 can be formed out of a flexible material (e.g., an elastic material) so that the body can curve to accommodate the different curved paths.

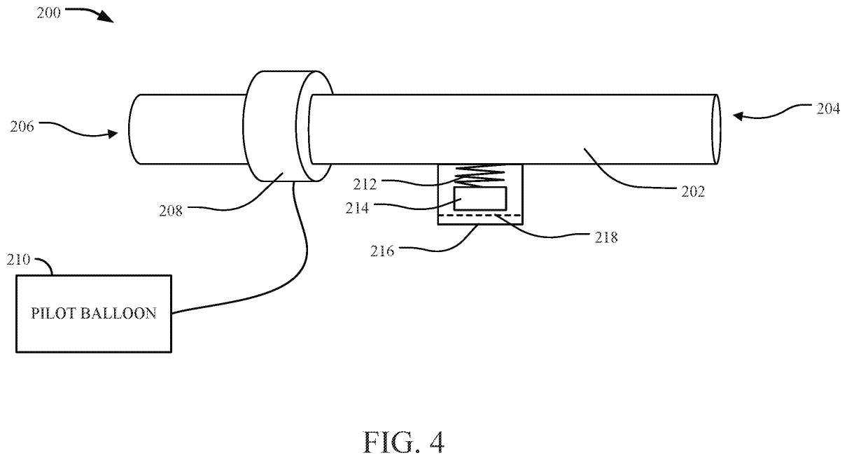

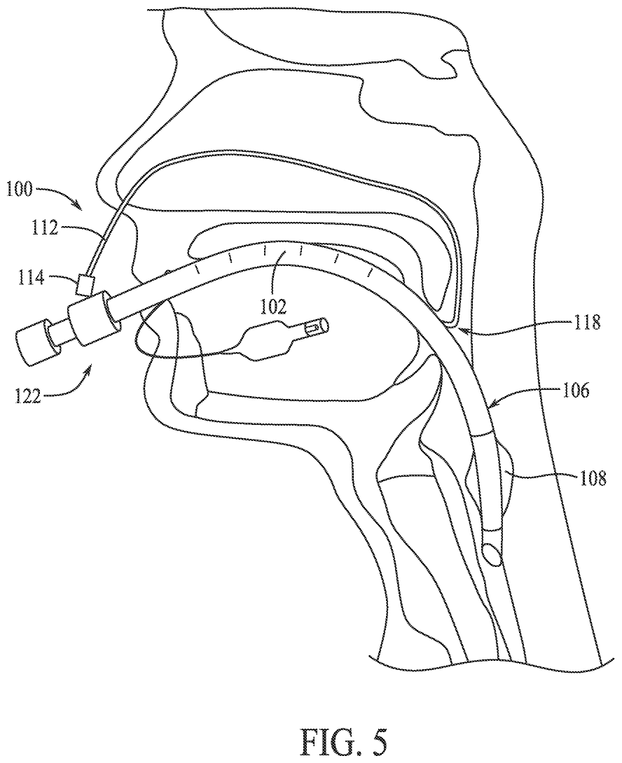

FIG. 4 shows a schematic illustration of an endotracheal tube 200, which can be similar to the endotracheal tube 100. Thus, the previous description of the endotracheal tube 100 also pertains to the endotracheal tube 200. Similarly to the endotracheal tube 100, the endotracheal tube 200 can include a tube 202 having a proximal end 204 and a distal end 206 opposite the proximal end 204, an inflatable cuff 208, a pilot balloon 210 in fluid communication with the inflatable cuff 208, a tether 212, and a magnet 214. In addition, the endotracheal tube 200 can include a pouch 216 that can contain the tether 212 and the magnet 214. As shown in FIG. 4, the tether 212 can be coupled to the tube 202, and the tether 212 can be coupled to the magnet 214. For example, one end of the tether 212 can be coupled to the tube 202 (e.g., using a fastener, such as an adhesive), and the other end of the tether 212 can be coupled to the magnet 214. Regardless of the configuration, the tether 212 can be coupled to the tube 202 and the magnet 214 can be coupled to the tether 212 so that the magnet 214 is separated from the tube 202.

In some cases, the pouch 216 can be coupled to the tube 202 or the pouch 216 can be integrally formed with the tube 202 (e.g., the pouch 216 and the tube 202 being formed of the same material and being a single monolithic component). Regardless, the tether 212 with the magnet 214 coupled thereto can be positioned within the internal volume of the pouch 216 so that the pouch partially (or entirely) encloses the magnet 214. In some cases, the pouch 216 can include a region of a material weakness 218, which can extend partially (or entirely) around the pouch 216. In some cases, the region of material weakness 218 can be a perforation, a tear, etc., and can generally facilitate removal of the magnet 214 from the pouch 216 and unraveling of the tether 212. For example, the tether 212 can be in a compact state (e.g., a coiled state), when, for example, the tether 212 and the magnet 214 are positioned within the pouch 216. Once the magnet 154 of the probe reaches the magnet 214 and the magnets 154, 214 are magnetically coupled together, the probe 150 can be retracted, which forces the magnet 214 against the wall of the pouch 216 thereby tearing the wall of the pouch 216 at the region of material weakness 218. At this point, the magnet 214 can be pulled away, via the magnet 154 of the probe 150, to unravel the tether 212 from the compact state (e.g., the coiled state) and move the magnet 214 (and a portion of the tether 212) out of the patient. In some embodiments, while the pouch 216 has been described as enclosing the magnet 214 and tether 212, in other configurations, the pouch 216 can have a hole. In this case, the magnet 214 can be advanced through the hole, which can prevent needing to tear the pouch 216.

In some embodiments, the endotracheal tube 200 can include a bar (not shown) that is coupled to the magnet 214 and the tube 202 so that the bar is positioned between the magnet 214 and the tube 202. The bar can include a region of material weakness (e.g., a portion of the bar being thinner than another region of the bar), which can facilitate breakage of the bar at the region of material weakness. For example, after the magnets 154, 214 are magnetically coupled together, the magnet 214 can be retracted (e.g., pulled) until the bar breaks in two (at the region of material weakness) thereby releasing the magnet 214 from the tube 202.

FIG. 5 shows a schematic illustration of the endotracheal tube 100 deployed within the patient, which can be applicable to the other endotracheal tubes described herein (e.g., the endotracheal tube 200). As shown in FIG. 5, the distal end 106 of the tube 102 and the inflatable cuff 108 are positioned within the trachea of the patient, while the proximal end 104 of the tube 102 is positioned outside of the patient. For example, the distal end 106 of the tube 102 can be inserted into the mouth of the patient, through the mouth of the patient, and into the trachea. Correspondingly, with the distal end 106 of the tube 102 positioned within the trachea, the proximal end 104 of the tube 102 can be positioned outside of the patient. As shown in FIG. 5, the tether 112 extends through the pharynx and through the nasal cavity so that a portion of the tether 112 is positioned outside of the patient. For example, after the magnet 154 of the probe 150 magnetically couples with the magnet 114, the probe 150 is retreated thereby advancing the magnet 114 through the pharynx, through the nasal cavity, and out the nasal cavity. In some cases, and as described above, the stop 118 blocks further advancement of the slide 116, which provides an anchor point for the tether 112 on the tube 102. In some embodiments, once the magnet 114 (and a portion of the tether 112) are positioned outside of the patient, the magnet 114 can be removed, and the tether 112 (which can sometimes include the magnet 114) can be coupled to an anchor 122 that is external to the patient. In some embodiments, the anchor 122 can include a clip that couples the tube 102 and the tether 112 together, that couples the tube 102, the tether 112, and a bridle tubing (not shown in FIG. 5) together, etc. In some configurations, the anchor 122 can include a tube holder (not shown in FIG. 5) that is secured around the head of the patient, and which couples to the tube 102. In this case, the tether 112 can also be secured to the tube holder.

As shown in FIG. 5, with a portion of the tether 112 positioned outside of the patient (and coupled to the anchor 122), the soft palate of the patient is positioned between the tube 102 and the tether 112. Accordingly, when the proximal end 104 of the tube 102 is advanced away from the patient, the tether 112 contacts the soft palate thereby blocking further advancement of the proximal end of the tube away from the patient. In this way, the tether 112 can help to ensure that the distal end 106 of the tube 102 is not undesirably removed from the trachea of the patient, by physically blocking the tube 102, via the tether 112. In addition, contacting of the soft palate with the tether 112 can be painful for the patient, and thus the patient is less likely to continue pulling the tube 102, which would cause additional pain for the patient. Thus, the tether 112 can act as a deterrent for pulling the tube 102 out from the patient.

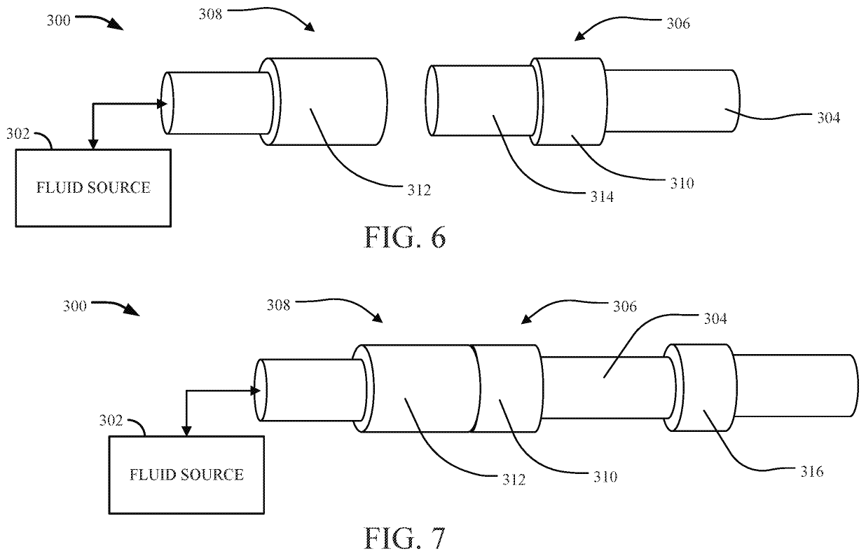

In some embodiments the endotracheal tube may be configured so that an external portion separates from the internal portion when a force is applied to the external portion, thereby preventing the internal portion from being removed (e.g., by a patient) at an inappropriate time. Accordingly, FIG. 6 shows a schematic illustration of an endotracheal tube system 300 with adapters separated from each other, while FIG. 7 shows a schematic illustration of an endotracheal tube system 300 with the adapters coupled together. The endotracheal tube system 300 can include a fluid source 302 (e.g., a ventilator), an endotracheal tube 304 including an adapter 306 (e.g., coupled to the proximal end of the tube of the endotracheal tube 304), and an adapter 308 that is in fluid communication with the fluid source 302. The adapter 306 is configured to be removably coupled to the adapter 308 (and vice versa). For example, the adapters 306, 308 can be magnetically coupled together, can be a snap-fit connection, can include a hook and loop fastener (e.g., one of the adapters including a hook fastener and the other adapter including a loop fastener), etc. In some cases, the adapters 306, 308 being magnetically coupled together can be advantageous in that not only can the magnets of the adapters 306, 308 can be configured to provide a relatively small coupling force (e.g., easily decoupled from each other the advantages of which are described below), but the magnets of the adapters 306, 308 can also guide a user to easily reconnect the adapters 306, 308 together (e.g., in the case bodily fluids including blood, vomit, etc., that obstruct the view of the adapters 306, 308 in the mouth of the patient). For example, the adapters 306, 308 can each include a respective magnet 310, 312, with the magnets 310, 312 having opposing poles that face each other. In other words, a north pole of the magnet 310 can face the south pole of the magnet 312 (or vice versa).

In some configurations, the adapter 306 can include an extension 314 that extends away from the tube of the endotracheal tube 304 (e.g., a distal end of the endotracheal tube 304), and the magnet 310. In some cases, the extension 314 can be inserted into the adapter 308 (e.g., so that the magnet 312 surrounds the extension 314), when the adapters 306, 308 are coupled together. Which can not only guide coupling of the adapters 306, 308 together, but can also help to better ensure a fluid tight seal between the adapters 306, 308. While the adapter 306 has been illustrated as including the extension 314, in other cases, the adapter 308 can include the extension 314, in which case the extension 314 is inserted into the adapter 306.

As shown in FIG. 7, the adapters 306, 308 are coupled together with the magnets 310, 312 being magnetically coupled together (e.g., and the magnets 310, 312 in contact with each other). In some embodiments, and advantageously, the force needed to decouple the adapters 306, 308 can be less than the force required to dislodge the inflatable cuff (or other balloon) that is positioned within the trachea of the patient. In this way, with the adapters 306, 308 coupled together, pulling of the adapter 308 away from the adapter 306 decouples the adapters 306, 308, rather than pulling the endotracheal tube 304, which would pull the distal end of the endotracheal tube 304 out of the trachea. Thus, the endotracheal tube 304 advantageously continues to be positioned within the trachea, even when the adapters 306, 308 are decoupled. In addition, the adapter 306 can be positioned within the mouth of the patient when the distal end of the endotracheal tube 304 is within the trachea, so that only the adapter 308 is positioned outside of the patient. In this way, when the patient pulls the adapter 308 (e.g., which is more likely to happen because the adapter 308 is more accessible to the patient), the adapters 306, 308 simply decouple and the distal end of the endotracheal tube 304 (e.g., including the inflatable cuff) does not move substantially away from the position of the distal end of the tube within the trachea prior to pulling the second adapter. Thus, when the adapters 306, 308 are decoupled, the distal end of the endotracheal tube 304 advantageously remains in the trachea of the patient.

In some embodiments, the endotracheal tube 304 can include a balloon 316 that can extend partially (or entirely) around the tube of the endotracheal tube 304. The balloon 316 can be selectively inflated and deflated, via a fluid source. For example, when the endotracheal tube 304 is deployed within the patient (e.g., the endotracheal tube 304 is positioned within the trachea of the subject), the balloon 316 can be positioned above the vocal cords. In this way, the balloon 316 can be inflated to contact a wall of the trachea thereby preventing the distal end of the endotracheal tube 302 from falling down further into the trachea, if, for example, the adapters 306, 308 decouple from each other (e.g., by the patient pulling on the adapter 308, or a component coupled to the adapter 308, such as other tubing).

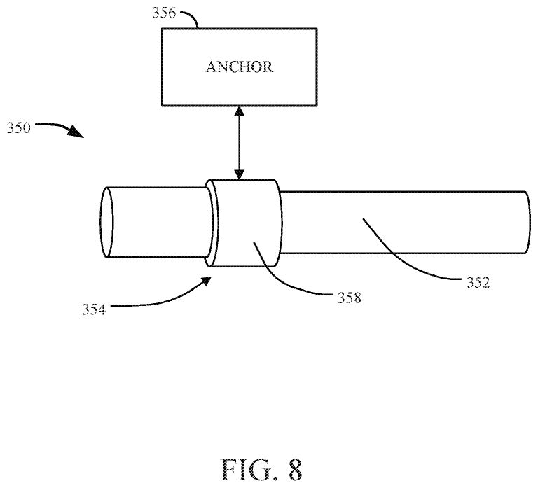

FIG. 8 shows a schematic illustration of an endotracheal tube system 350, which relates to the other endotracheal tube systems described herein. Thus, the endotracheal tube systems described herein pertain to the endotracheal tube system 350 (and vice versa). The endotracheal tube system 350 can include an endotracheal tube 352 including an adapter 354 (e.g., coupled to a proximal end of the endotracheal tube 352), and an anchor 356. The endotracheal tube 352 can be coupled to (e.g., removably coupled to) the anchor 356, and in particular, the adapter 354 can be coupled to (e.g., removably coupled to) the anchor 356. For example, including when the adapter 354 includes a magnet 358, the anchor 356 can also include a magnet that magnetically couples to the magnet 358.

The anchor 356 can be implemented in different ways. For example, the anchor 356 can be positioned outside of the patient and can be coupled to the outside portion of the patient. For example, the anchor can be fastened around an anatomical structure of the patient, including, for example, the head of the patient. In some cases, the anchor 356 can include a tube holder (e.g., a Hollister tube holder, an endotracheal tube fastener, etc.) that couples to the patient (e.g., by adhering to an external surface of the patient). In some embodiments, the anchor 356 can include a clip that couples together the endotracheal tube 352 (including the tube of the endotracheal tube 352, a tether of the endotracheal tube 352, etc.), a bridle tube, etc. In some cases, when the anchor 356 includes a clip and a tube holder, the clip can couple the tube holder, the tube of the endotracheal tube 352, the tether of the endotracheal tube 352, and a bridle tube together. In some cases, the clip can be a frictional clip (e.g., a snap-fit clip), a magnetic clip, etc. In some configurations, when the clip is a frictional clip, the clip can better secure the components together.

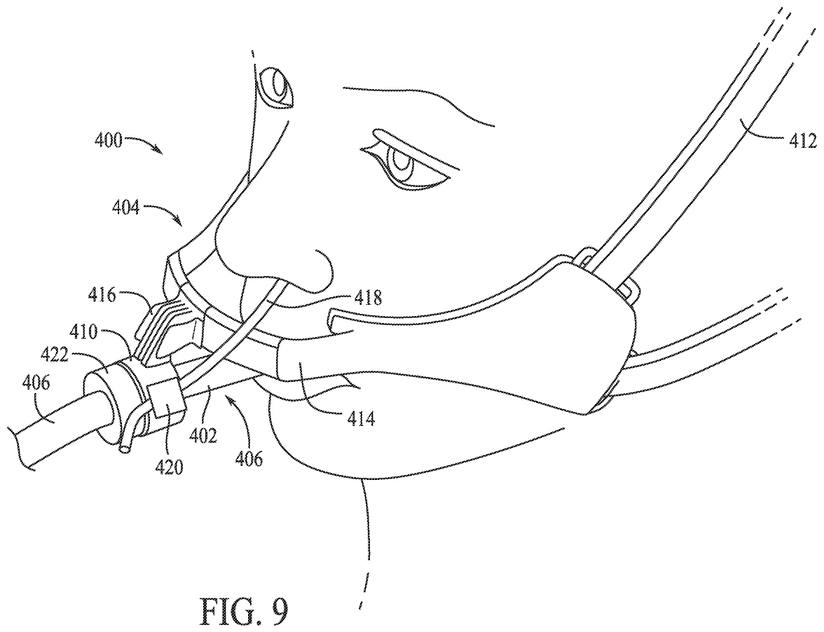

FIG. 9 shows a schematic illustration of an endotracheal tube system 400, which relates to the other endotracheal tube systems described herein. Thus, the endotracheal tube systems described herein pertain to the endotracheal tube system 400 (and vice versa). The endotracheal tube system 400 can include an endotracheal tube 402, a tube holder 404, and a ventilator tube 406. The endotracheal tube 402 can include a tube 408, and an adapter 410 coupled to the tube 408. The tube holder 404 can be positioned outside of the patient and can be coupled to an exterior surface of the patient. For example, the tube holder 404 can include one or more straps 412 that can extend around the head of a patient to secure the tube holder 404 to the head of the patient. In some cases, the tube holder 404 can include a bracket 414 that can be coupled to the one or more straps 412 and can extend around a portion of the face of the patient. In some cases, the bracket 414 can be positioned below a nose of the patient, and above the mouth of the patient (e.g., when the tube holder 404 is secured to the patient). As shown in FIG. 9, the tube holder 404 can be coupled to the endotracheal tube 402, which can include the tube holder 404 being removably coupled to the endotracheal tube 402 (e.g., being snap fitted, being magnetically coupled, etc.). For example, the tube holder 404 can include an arm 416 that can be coupled to the bracket 414 at one end of the arm 416, and can be coupled to the adapter 410 of the endotracheal tube 402 at the opposing end of the arm 416. In some cases, the arm 416 can be removably coupled to the adapter 410 (e.g., the arm 416 being magnetically coupled to the arm 416). In this way, after the endotracheal tube 402 has been deployed, the tube holder 404 can be secured around the patient, and the arm 416 of the tube holder 404 can be coupled to the adapter 410 of the endotracheal tube 402 to secure the endotracheal tube 402 (e.g., to prevent inadvertent pulling out of the endotracheal tube 402).

As shown in FIG. 9, the endotracheal tube 402 can include a tether 418, and a magnet 420 that is coupled to the tether 418. In some cases, after the endotracheal tube 402 has been deployed and the magnet 420 has been advanced through and out the nasal passage of the patient, the magnet 420 can be coupled to the adapter 410 (e.g., with the magnet 420 being removably coupled to the adapter 410). In this way, the tether 418 can be prevented from being retreated back through the nasal passage of the patient. In other configurations, the tether 418 can be coupled to the endotracheal tube 402 (e.g., can be tied around the adapter 410). In some embodiments, the ventilator tube 406 can include an adapter 422 that can be removably coupled to the adapter 410 of the endotracheal tube 402. For example, after the endotracheal tube 402 has been deployed, the magnet 420 retrieved, and the tube holder 404 secured to the patient, the adapter 422 of the ventilator tube 406 can be coupled to the adapter 410 of the endotracheal tube 402. In this way, with the adapters 410, 422 coupled together, the ventilator tube 406 can provide gas (e.g., air) to and through the endotracheal tube 402, and the if the patient inadvertently pulls on the ventilator tube 406, the ventilator tube 406 decouples from the endotracheal tube 402 at the adapters 410, 422, without the endotracheal tube 402 being pulled with the ventilator tube 406. This can be advantageous in that the risk for inadvertent pulling out of the endotracheal tube 402 can be significantly mitigated.

The present disclosure has described one or more preferred embodiments, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular embodiments or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or embodiments. Further, references to particular rotational or other movements (e.g., counterclockwise rotation) is generally intended as a description only of movement relative a reference frame of a particular example of illustration.

Certain operations of methods according to the disclosure, or of systems executing those methods, may be represented schematically in the figures or otherwise discussed herein. Unless otherwise specified or limited, representation in the figures of particular operations in particular spatial order may not necessarily require those operations to be executed in a particular sequence corresponding to the particular spatial order. Correspondingly, certain operations represented in the figures, or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use embodiments of the disclosure. Various modifications to the illustrated examples will be readily apparent to those skilled in the art, and the generic principles herein can be applied to other examples and applications without departing from the principles disclosed herein. Thus, embodiments of the disclosure are not intended to be limited to embodiments shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Various features and advantages of the disclosure are set forth in the following claims.

What is claimed is:

1. An endotracheal tube comprising:
a tube having a distal end that is configured to be inserted into the trachea of a patient and a proximal end that is configured to be positioned within the mouth of the patient or outside of the patient;
a tether having a first end coupled to the tube; and
a magnet coupled to the tether, the magnet being configured to be advanced through the pharynx, through and out the nasal cavity thereby positioning the soft palate of the patient between the tube and the tether.

2. The endotracheal tube of claim 1, wherein when the proximal end of the tube is advanced away from the patient, the tether contacts the soft palate to block further advancement of the proximal end of the tube away from the patient.

3. The endotracheal tube of claim 1, further comprising:
a channel that extends along the tube;
a slide coupled to a second end of the tether opposite the first end; and
a stop coupled to the tube,
wherein the magnet is configured to be moved along the channel in a first direction towards the proximal end of the tube until the slide contacts the stop thereby blocking further advancement of the magnet in the first direction.

4. The endotracheal tube of claim 1, wherein a second end of the tether opposite the first end is configured to be coupled to an anchor that is external to the patient thereby fixing the position of the endotracheal tube within the patient.

5. The endotracheal tube of claim 4, wherein the anchor wraps around the head of the patient, and
wherein a portion of the tube is coupled to the anchor.

6. The endotracheal tube of claim 1, wherein the magnet is removably coupled to the tether.

7. The endotracheal tube of claim 1, further comprising a first adapter coupled to the proximal end of the tube,
wherein a second adapter of a conduit in fluid communication with a ventilator is configured to be removably coupled to the first adapter, and
wherein the first adapter is configured to be positioned within the mouth of the patient when the distal end of the tube is placed in the trachea of the patient.

8. The endotracheal tube of claim 7, wherein when the second adapter is pulled away from the first adapter of the tube, the second adapter decouples from the first adapter.

9. The endotracheal tube of claim 7, wherein the magnet is a first magnet,
wherein the first adapter comprises a second magnet,
wherein the second adapter comprises a third magnet, and
wherein the first adapter magnetically couples to the second adapter, via the interaction between the second magnet and the third magnet.

10. The endotracheal tube of claim 7, further comprising an inflatable cuff coupled to the tube and configured to be positioned within the trachea of the patient, and
wherein when the first adapter is coupled to second adapter, and the second adapter is pulled away from the first adapter, the second adapter decouples from the first adapter with the distal end of the tube remaining in the trachea.

11. The endotracheal tube of claim 1, wherein the magnet is a first magnet, and
wherein a probe with a second magnet coupled thereto is configured to be inserted into the nasal cavity, through the nasal cavity, into the pharynx to be magnetically coupled to the first magnet, and
wherein with the second magnet magnetically coupled to the first magnet, the probe is configured to be retreated back through and out the nasal cavity.

12. A method for deploying an endotracheal tube comprising:
inserting a distal end of a tube into the trachea of a patient;
positioning a proximal end of the tube within the mouth of the patient or outside of the patient,
a first end of a tether being coupled to the tube, and
a magnet being coupled to the tether; and
advancing the magnet through the pharynx of the patient, through and out the nasal cavity such that the soft palate of the patient is positioned between the tube and the tether.

13. The method of claim 12, wherein, when the proximal end of the tube is advanced away from the patient, the method further comprises:
contacting the soft palate with the tether to block further advancement of the proximal end of the tube away from the patient.

14. The method of claim 12, further comprising:
providing a channel that extends along the tube, wherein a slide is coupled to a second end of the tether opposite the first end and a stop is coupled to the tube, and
moving the magnet along the channel in a first direction towards the proximal end of the tube until the slide contacts the stop to block further advancement of the magnet in the first direction.

15. The method of claim 12, further comprising:
coupling a second end of the tether opposite the first end to an anchor that is external to the patient to fix the position of the endotracheal tube within the patient.

16. The method of claim 15, further comprising:
wrapping the anchor around the head of the patient, and
coupling a portion of the tube to the anchor.

17. The method of claim 12, wherein the magnet is removably coupled to the tether.

18. The method of claim 12, further comprising
providing a first adapter coupled to the proximal end of the tube,
wherein a second adapter of a conduit in fluid communication with a ventilator is configured to be removably coupled to the first adapter, and
positioning the first adapter within the mouth of the patient when the distal end of the tube is placed in the trachea of the patient.

19. The method of claim 18, wherein, when the second adapter is pulled away from the first adapter of the tube, the method further comprises:
decoupling the second adapter from the first adapter.

20. The method of claim 18, wherein the magnet is a first magnet,
wherein the first adapter comprises a second magnet,
wherein the second adapter comprises a third magnet, and
wherein the method further comprises:
magnetically coupling the first adapter to the second adapter based on the interaction between the second magnet and the third magnet.

21. The method of claim 18, further comprising:
providing an inflatable cuff coupled to the tube,
positioning the inflatable cuff within the trachea of the patient,
coupling the first adapter to the second adapter, and
decoupling, when the second adapter is pulled away from the first adapter, the second adapter from the first adapter,
wherein the distal end of the tube remains in the trachea.

22. The method of claim 12, wherein the magnet is a first magnet, and
wherein the method further comprises:
inserting a probe with a second magnet coupled thereto into the nasal cavity, through the nasal cavity, and into the pharynx,
magnetically coupling the second magnet to the first magnet, and
retreating the probe with the second magnet magnetically coupled to the first magnet back through and out the nasal cavity.
